(19) United States
(12) Patent Application Publication
(10) Pub. No.: US 2025/0072755 A1
(43) Pub. Date: Mar. 6, 2025

(54) SYSTEM FOR LOCALISING LIGHT IN LIGHT-SCATTERING MEDIA

(71) Applicant: DEEP LIGHT VISION AB, Lund (SE)

(72) Inventors: Stefan KRÖLL, Dalby (SE); David HILL, Lund (SE); Alexander BENGTSSON, Lund (SE); Lars RIPPE, Lund (SE); Emilie KRITE SVANBERG, Lund (SE)

(73) Assignee: DEEP LIGHT VISION AB, Lund (SE)

(21) Appl. No.: 18/703,310

(22) PCT Filed: Oct. 24, 2022
(86) PCT No.: PCT/EP2022/079495

§ 371 Date: Apr. 19, 2024

(30) Foreign Application Priority Data
Oct. 24, 2021 (SE) 2151298-3

(51) Int. Cl. A61B 5/00; A61B 5/05

(52) U.S. Cl. CPC A61B 5/0059; A61B 5/0051; A61B 5/05; A61B 2562/0238

(57) ABSTRACT

A system for light measurements in a subject, the system comprising at least one ultrasound transducer (13) configured for directing an ultrasound field with an ultrasound frequency to a location (15) inside a tissue site of the subject; at least one laser (12) configured for emitting light within a wavelength-range of infrared (IR), visible or ultraviolet light, and for directing light of a certain frequency to the tissue site; at least one optical filter (14) arranged before at least one light detector, the filter configured for suppressing the incident light frequency, but for transmitting light shifted by the ultrasound frequency to be detected by the light detector; and at least one light-reflecting member (16,17) configured to be arranged on a surface of the tissue site of the subject.

### BACKGROUND OF THE INVENTION

### Field of the Invention

This disclosure pertains in general to a device and method to selectively attain light deep inside a light scattering medium, such as biological tissue. More particularly, the disclosure relates to a device and method to achieve more localized measurements using light deep inside a scattering medium than can be achieved by conventional methods. Especially, the disclosure relates to the use of acoustic-frequency-shifted laser light and optical components to achieve localized light measurements deep inside a scattering medium.

### Background of the Disclosure

The ability to shine light into turbid, i.e., light scattering, media is important in many settings, including but not limited to biomedical applications. Optical imaging provides highly sensitive molecular contrast in a simple and non-invasive manner. Traditional light microscopy relies on penetration of light through shallow layers, in the order of tens of micrometres. Confocal microscopy techniques extend the penetration depths up to in the order of 0.1 mm, while optical coherence tomography can reach to depth in the order of 0.5 mm. This is approximately the limit of high-resolution optical imaging modalities, which rely on the suppression of diffusely scattered light. At deeper depths, diffuse light scattering dominates the interaction of the light field and the medium. The attenuation of an impending light beam at a certain depth is largely determined by the scattering of the medium. This fundamental aspect means that even at the most favourable wavelengths, the maximum penetration depth where light is detectable is limited to in the order of 10 cm in typical biological tissue.

In the diffuse scattering regime, from about a millimetre up to several centimetres, other optical analysis and optical imaging modalities have been developed. One example is pulse oximetry for determination of blood oxygen saturation. One example of optical imaging is diffuse optical tomography, which can be performed, for example, with endogenous tissue contrast, with added contrast agents, in fluorescence, or with added fluorescing agents. Generally, because of the diffuse scattering, spatial localization is limited with these methods to in the order of several millimetres up to a centimetre or more. In addition, because of the very low light intensities in the detected light, the detection schemes are often cumbersome, slow, and require advanced and expensive equipment.

To improve the spatial localization when probing turbid media with light, photoacoustic schemes have been devised. In photoacoustic tomography, acoustic waves that emanate locally, due to slight heating in the tissue due to absorption of laser pulses, are detected by ultrasound transducers. Photoacoustic tomography takes advantage of the fact that ultrasound waves are scattered several orders of magnitude less than light waves in tissue. Thus, the spatial localisation of the origin of the acoustic waves can be in the order of a millimetre or less.

Another example of the use of acoustics to improve localisation of light interaction in tissue is the use of acoustic photon tagging, as described for example in J. Gunther & S. Anderson-Engels (2017, October), Review of current methods of acousto-optical tomography for biomedical applications, Frontiers of Optoelectronics, 10(3), 211-238. By insonifying tissue, and directing laser light into the tissue, only the laser light that passes through the region occupied by an acoustic field will be frequency-shifted by the acoustic frequency. By optical detection of the frequency-shifted light only, it can be known that that light has interacted with the acoustic field, which compared to the light can be made to occupy small and localized volume.

Another example of the use of acoustic is described by M. Kempe et al, Acousto-optic tomography with multiply scattered light, Vol. 14, No. 5/May 1997/J. Opt. Soc. Am. A. A further example is described in US20060253007 A1 where ultrasound waves overlaps optical spectroscopy. The output from the ultrasound transducer is used to o physically modulate (vibrate) the selected target using ultrasound radiation pressure by focus the ultrasound into the target at various modulation frequencies. However, although photoacoustic methods can provide better spatial resolution than pure optical methods, these methods still suffer from the limitation that the light does not easily penetrate deep into light-scattering media, due to the strong attenuation provided by the scattering.

As described above, previously known methods to perform light measurements inside a light-scattering medium have a limited penetration depth and/or poor spatial resolution. Hence, new improved apparatus and methods for deep light penetration in light scattering media would be advantageous.

### SUMMARY OF THE INVENTION

Accordingly, examples of the present disclosure preferably seek to mitigate, alleviate or eliminate one or more deficiencies, disadvantages or issues in the art, such as the above-identified, singly or in any combination by providing a device, system or method according to the appended patent claims for selectively attaining high light intensities deep inside scattering media.

The disclosure generally comprises an optical part and an acoustic part. In an example, in the schematic illustration in FIG. 1, the system 100 comprises an optical part of the system where 12 is a laser, and 14 is an optical filter. The acoustic part of the system comprises 13 which is an ultrasonic device. The ultrasonic device emits an acoustic field into the light-scattering medium 11. The position of the acoustic field is 15. The laser 12 may be directed into the medium 11 via the input aperture(s) 19, and the light 18 is diffusely scattered in the medium. Part of the light 18c will pass through the ultrasound field at position 15, and part of that light may be frequency-shifted through interaction with the ultrasound field. Since the optical frequency of the laser light is many orders of magnitude higher than the frequency of the ultrasound, the frequency shift corresponds to a small wavelength difference in the laser light. The frequency-shifted light may propagate from the position 15 diffusely through the medium, and part of it may leave the medium via the output aperture(s) 20 and hit the optical filter 14. The optical filter 14 may largely remove the original laser frequency, leaving substantially the frequency-shifted light, which can be detected. The frequency-shifted light carries information only from the position 15, and thus this setup may provide spatial information inside the medium 11. By scanning the position 15, it may be possible to attain a map or image of the optical contrast inside the medium 11, such as information about the oxygen saturation in tissue. Such an image taken with an embodiment of system 100 is illustrated in FIG. 2.

Around the laser injection point, there may be a light-reflecting member 16, which may reflect a substantial part of any light that escapes the medium 11. A similar light-reflecting member 17 may be arranged around the point where the frequency-shifted light exits the medium to the optical filter.

The light-reflecting member 16 has the function to prevent part of the laser light from escaping the medium 11 before said laser light has had a chance to interact with the ultrasound field at 15. Thus, the light-reflecting member 16 may serve to increase the total light fluence in the tissue, including point 15, which may increase the amount of frequency-shifted light. This may result in an increased signal.

Similarly, the light-reflecting member 17 may have the function to prevent part of the frequency-shifted light from escaping the medium 11 other than through the output aperture 20 and thus may increase the amount of light passed through the filter 14. This may also result in an increased signal.

The optical filter 14 may be comprised of a slow-light filter, example given in FIGS. 3 and 4. This filter may be comprised of a host crystal 21 doped with ions 22 with strong optical absorption at the original frequency of the laser 23. As the ions replace other atoms in the host crystal, they may slightly distort its crystal lattice 21. While the individual absorption of these ions is strong and narrow in frequency, the entire ensemble of doped ions may see different crystal fields and thus slightly change at what frequency they absorb in relation to each other. In FIG. 3, three different ion classes 22 are depicted which have different energy separations between their ground and excited states, as seen in FIG. 3b). The multitude of different ion classes yield a total absorption profile of the ensemble of doped ions in the host crystal that may be two to three orders of magnitude wider in frequency than any frequency shift induced by the acoustic field.

In this absorption profile it may be possible to construct different enduring spectral structures by optical pumping techniques. This optical pumping may be performed with or without the application of electric and/or magnetic fields to split energy levels of the ions. An example of a resulting filter and the crystal absorption profile is seen in FIG. 4, where the absorption may be unaffected or higher for the original laser frequency light 23 while any absorption may be removed for the frequency-shifted light 24 which can proceed to a detector 25. A side effect of this modified absorption profile is that the frequency shifted light may experience a reduction in group velocity by several orders of magnitude. As such, the signal 24 may be additionally differentiated from the original laser light 23 via time gating. It should be emphasized that the term “light” when used in this specification is not limited to light visible to the human eye, but also comprises wavelengths in the ultraviolet and infrared regions.

It should also be emphasized that the term “comprises/comprising” when used in this specification is taken to specify the presence of stated features, integers, steps or components but does not preclude the presence or addition of one or more other features, integers, steps, components or groups thereof.

### BRIEF DESCRIPTION OF THE DRAWINGS

These and other aspects, features and advantages of which examples of the disclosure are capable of will be apparent and elucidated from the following description of examples of the present disclosure, reference being made to the accompanying drawings, in which

FIG. 1 is illustrating example of schematic setup for imaging using acoustic photon tagging.

FIG. 2 is an image taken of an optically absorbing inclusion in using an example of a system of FIG. 1.

FIG. 3 is illustrating a schematic crystal with 3 different classes of doped ions. Such a crystal may be used to create a slow light filter.

FIG. 4 is illustrating a schematic slow-light filter. The absorption of the ion classes in the crystal have been manipulated to allow for passage of the frequency-shifted light (full) while the original laser frequency (dashed) is absorbed.

FIG. 5 is illustrating a schematic transmission mode experimental setup with a homogeneous medium.

FIG. 6. is illustrating a frequency shifted signal strength when using/not using a reflecting member at different depths.

FIG. 7 is illustrating a frequency shifted signal strength when using/not using a reflecting member for a single signal at a given depth.

FIG. 8 is illustrating the effect of the size of the reflecting member on the acoustic photon tagging signal strength.

FIG. 9 is illustrating a schematic reflection mode experimental setup for imaging an absorbing inclusion.

FIG. 10 is illustrating the image taken of an absorbing inclusion with (a) and without (b) a reflecting member covering the input side.

FIG. 11 is illustrating the effect of the reflecting member on the ability to differentiate oxygenation levels when using/not using a reflecting member.

FIG. 12 is illustrating that there are optimal design configurations when using acoustic photon tagging to image tissue oxygenation.

FIG. 13 is schematically illustrating how a hollow core light guide improves the amount of light that is guided to the crystal inside the cryostatic device.

### DESCRIPTION OF EXAMPLES

The following disclosure focuses on examples of the present disclosure applicable to improve localisation of light interaction in light-scattering media. The disclosure may be applicable for measurements on a subject, such as a human or animal. The disclosure may be applicable for measurements of oxygen saturation deep inside living tissue, such as the brain, heart, female breast, or muscle tissue. However, it will be appreciated that the description is not limited to this application but may be applied to many other systems where localisation of light interaction in light-scattering media is useful.

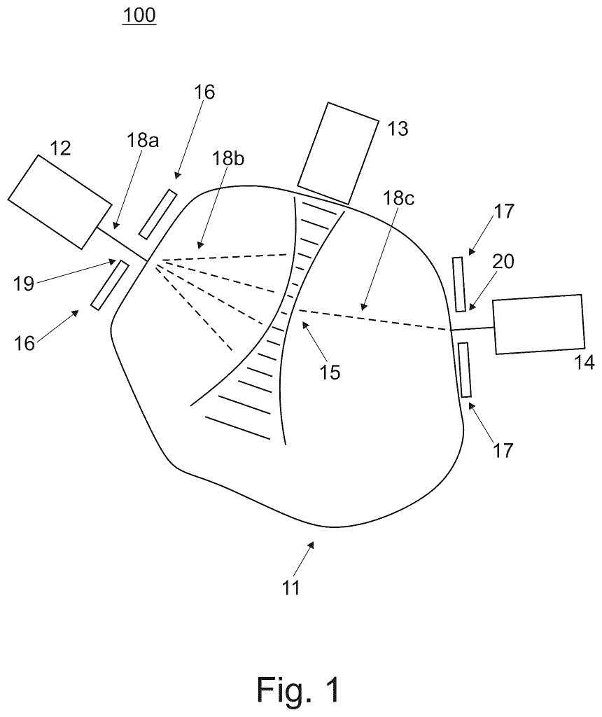

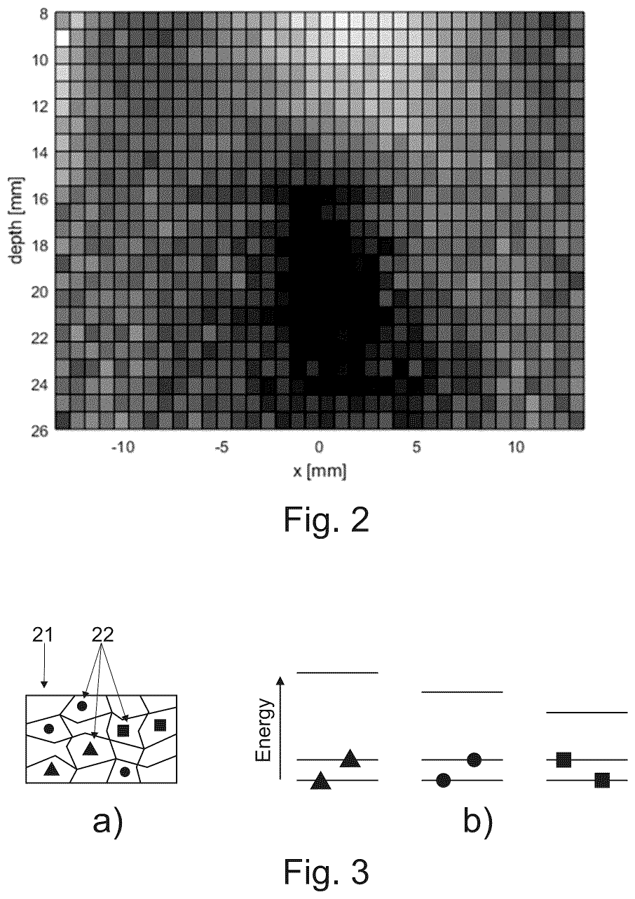

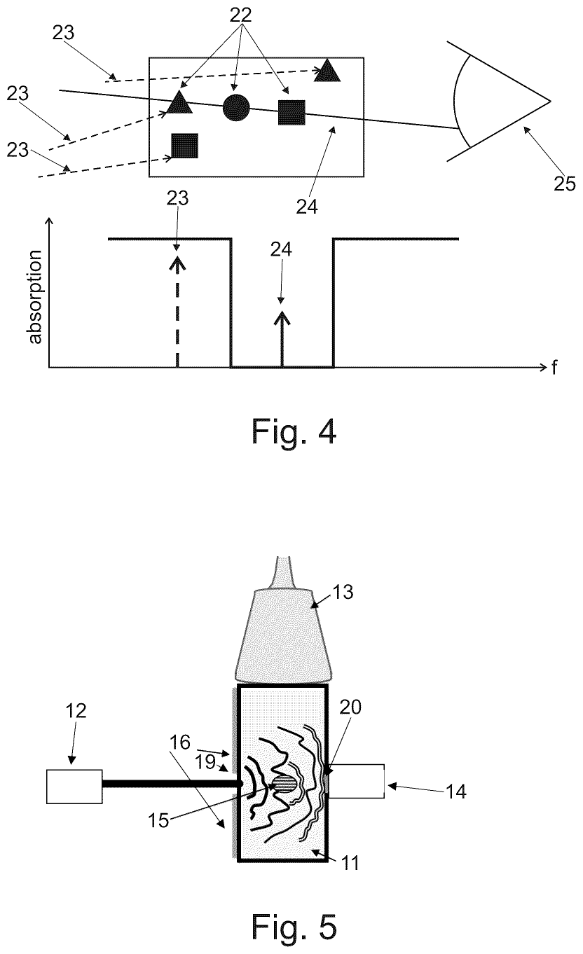

The disclosure generally comprises an optical part and an acoustic part. In an example, in the schematic illustration in FIG. 1, the system 100 comprises an optical part of the system which includes a light source 12, such as a laser, and an optical filter 14. The acoustic part of the system includes an ultrasonic device 13. The ultrasonic device 13 emits an acoustic field into the light-scattering medium 11 at in order to occupy the point 15. A light beam 18a emitted from the laser 12 is directed into the medium 11, and the light beam 18a is diffusely scattered 18b in the medium. Part of the diffused light 18b may pass through the position occupied by the ultrasound field 15, and may be frequency-shifted through interaction with the ultrasound field. The ultrasound frequency may be in the order of 10⁶ Hz, while the optical frequency may be in the order of 10¹⁴ Hz. Since the optical frequency of the laser light is many orders of magnitude higher than the frequency of the ultrasound, the frequency shift corresponds to a small wavelength difference in the laser light, which may be in the order of a few femtometers. The frequency-shifted light may propagate from the position occupied by the ultrasound field 15 diffusely through the medium, and part of the frequency-shifted light 18c may leave the medium and hit the optical filter 14. The optical filter 14 largely removes the original laser frequency, leaving substantially the frequency-shifted light, which can be detected. The optical filter 14 needs to have special properties to suppress the carrier frequency at around 10¹⁴ Hz, when the frequency-shifted light is only separated by in the order of 10⁶ Hz. It also needs to be largely independent of the angle of the incoming light, which is diffuse. This performance can be realised as a so-called slow-light filter, as described in H. Zang et al (2012, March), Slow light for deep tissue imaging with ultrasound modulation, Appl. Phys. Lett. 100(13), 131102. The frequency-shifted light carries information only from the position 15, and thus this setup provides spatial information inside the medium 11. By scanning the position 15, representing the volume occupied by the ultrasound, it is possible to attain a map or image of the optical contrast inside the medium 11, such as information about the oxygen saturation in tissue. The lateral scanning may be performed either by mechanical movement of the ultrasound source or by controlling the individual elements of the ultrasound source to move the ultrasound field electronically. The longitudinal scanning is performed by timing an optical pulse to different depth positions of the ultrasonic pulse. An experimentally obtained image generated in this manner is seen in FIG. 2, where position 15 has been scanned over an area of high absorption, i.e. low oxygen saturation for wavelengths shorter than 800 nm. In order to extract information about the oxygen saturation, it is preferable to perform the measurement at least two different wavelengths. For example, it is preferable to have one wavelength around 800 nm, and one wavelength in the interval 600-770 nm. The spectral absorption profiles of oxygen-saturated haemoglobin and non-oxygen-saturated haemoglobin are such that the light absorption is similar at around 800 nm, while different at wavelengths below or above 800 nm. Alternatively, one can choose one wavelength at around 800 nm, and another wavelength in the interval 820-1200 nm. By comparing the signal at the two wavelengths, it is possible to deduce the ratio of oxygen-saturated to non-oxygen-saturated haemoglobin. Note that the method is not limited to two wavelengths. In some situations, it may be preferable to use more than two wavelengths, e.g., to increase the accuracy when determining the oxygen saturation.

To substantially improve the signal levels, it is advantageous to add light-reflecting members around the laser injection position and/or the light filter exit position. Again, referring to FIG. 1, around the laser injection point, there is a first light-reflecting member 16, which reflects a substantial part of any light that escapes the medium 11. A similar second light-reflecting member 17 may be arranged around the point where the frequency-shifted light exits the medium to the optical filter.

The first light-reflecting member 16 has the function to prevent part of the laser light from escaping the medium 11 before said laser light has had a chance to interact with the ultrasound field at point 15. Thus, the first light-reflecting member 16 may serve to increase the total light fluence at point 15, which increases the amount of frequency-shifted light. This may result in an increased signal.

Similarly, the second light-reflecting member 17 has the function to prevent part of the frequency-shifted light from escaping the medium 11 before said frequency-shifted light has had a chance to exit and be collected by the optical filter 14. Thus, the second light-reflecting member 17 may serve to increase the amount of frequency-shifted light that is collected by the optical filter 14. This may also result in an increased signal.

The addition of a light-reflecting member at the surface of the light-scattering medium, as described here, is advantageous in the case of photo-acoustic methods, since with photo-acoustic methods, there is no requirement to spatially localise the primary laser light. This contrasts with for example diffuse optical tomography, where adding a light-reflecting member at the surface of the light-scattering medium will increase the light fluence inside the light-scattering medium, but it may at the same time decrease the spatial localisation of the light, which is an important property in diffuse optical tomography.

The light-reflecting member may be made of any suitable material that has high reflectivity at the relevant wavelengths. This includes, but is not limited to, glass or metal mirrors, metal surfaces, plastics with high reflectivity, or surfaces covered with reflective paint. Surfaces with a reflectivity above approximately 80% would be considered highly reflective in this context, but it should be noted that the addition of any reflective surface is advantageous, even at lower reflectivity values.

To demonstrate the advantage of using a light-reflecting medium at the surface of the light-scattering medium, experiments were performed. The experimental setup is depicted in FIG. 5. An ultrasound pulse with center frequency f_{US} was generated from an ultrasound transducer 13, and said ultrasound pulse propagated into a phantom tissue slab 11 with known optical properties. When the ultrasound field reached point 15, a short light pulse from a narrow frequency laser 12 with frequency f_{C} was fired at the slab into a point sized input aperture 19. This initial light may be denoted carrier light. The carrier light diffused into the ultrasound pulse and a part of it was frequency-shifted to the frequency f_{S}=f_{C}+f_{US} due to the acousto-optical effect. This ultrasound shifted light may be denoted tagged light. Both light fields were then collected opposite to the input side by with a single 1 cm diameter output aperture 20. The tagged light was separated from the carrier light using a slow light filter and detected on a photomultiplier tube, collectively denoted 14. A reflecting film with reflectivity R=0.98 was used as a reflecting member 16 and placed over the input side with a 2.6 mm diameter hole in it for transmitting the carrier light and the signal light was measured again. The signal light was measured with and without film for different thicknesses of the slab.

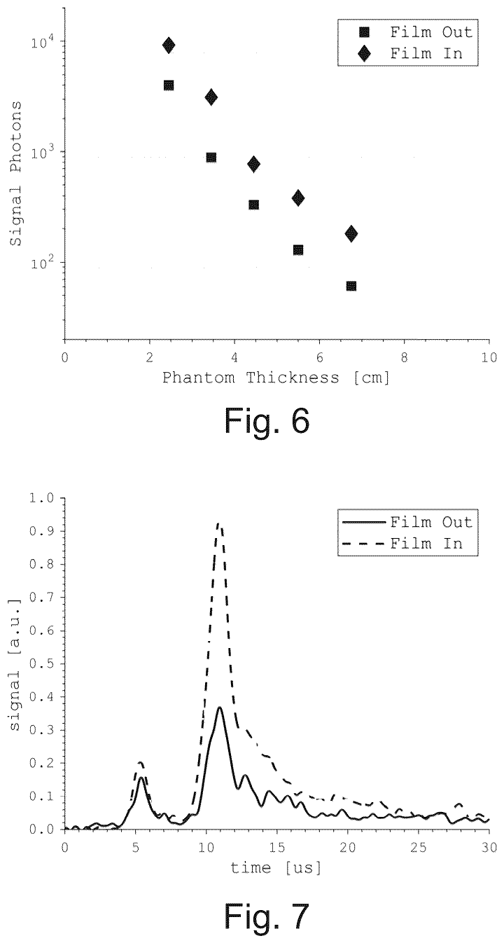

The amount of detected photons as a function of phantom thickness can be seen in FIG. 6. FIG. 7 shows the signal in time averaged over 1000 shots for a phantom of thickness 6.7 cm. Both FIGS. 6 and 7 indicate that the signal strength is improved between two and three times, with a larger effect at deeper imaging depths.

To further demonstrate the advantage of using a light-reflecting member at the surface of the light-scattering medium 11, simulations were performed. The use of the diffusion equation to calculate light fluence, F, in light-scattering media is well known in the art. The diffusion equation can be stated as:

$$a * F - D * div ( grad ( F ) ) = S \qquad ( 1 )$$

Where D=1/(3(a+c)) is the diffusion constant, a is the absorption coefficient and c the reduced scattering coefficient and S describes the source. The light that can be detected by a detector is described by the photon flux J impending on the detector surface. The flux by Fick's law:

$$J = - D * grad ( F ) \qquad ( 2 )$$

Boundary conditions for the light diffusion equation are

$$F + 2 * A * D * ( n \vert grad ( F ) ) = 0 \qquad ( 3 )$$

Where A is a constant which given by the average reflectance of light at the interface with normal vector n between the tissue and the surrounding medium (e.g. air or reflecting member). The diffusion equation can be solved by, e.g., the finite element method.

To evaluate the effect of the light-reflecting medium on the surface of the light-scattering medium, it is useful to define the contrast-to-noise ratio (CNR) when measuring the oxygen saturation of a tissue. In short, the CNR is a measure of how detectable a change of the tagged light is when moving position 15 between two close points which locally have different optical properties. These optical properties depend on the blood oxygenation of the tissue. The CNR for N laser pulse shots per detection position is defined as:

$$CNR = sqrt ( N ) * \vert ST 1 - ST 2 \vert / sqrt ( ST 1 + TF * SC ) \qquad ( 5 )$$

Where ST1 and ST2 is the detected shifted light from two different areas during the position 15 scan. SC is the detected signal light and TF is the filter transmission for the carrier light. For example, for a filter suppression of 80 dB, TF=10⁻⁸.

The simulations were carried out using the commercial FEM-solver COMSOL. A slab geometry with varying thicknesses was simulated with or without a reflecting boundary on one side. This reflecting boundary had a 2.6 mm diameter hole in it (same as in the experimental setup). The detected carrier light was simulated by the flux into light guide on the opposite side generated by a point source at the depth 1/c beneath the film hole. To verify the simulations, the carrier light signal was compared with the experimental results, and the data corresponded in the two cases.

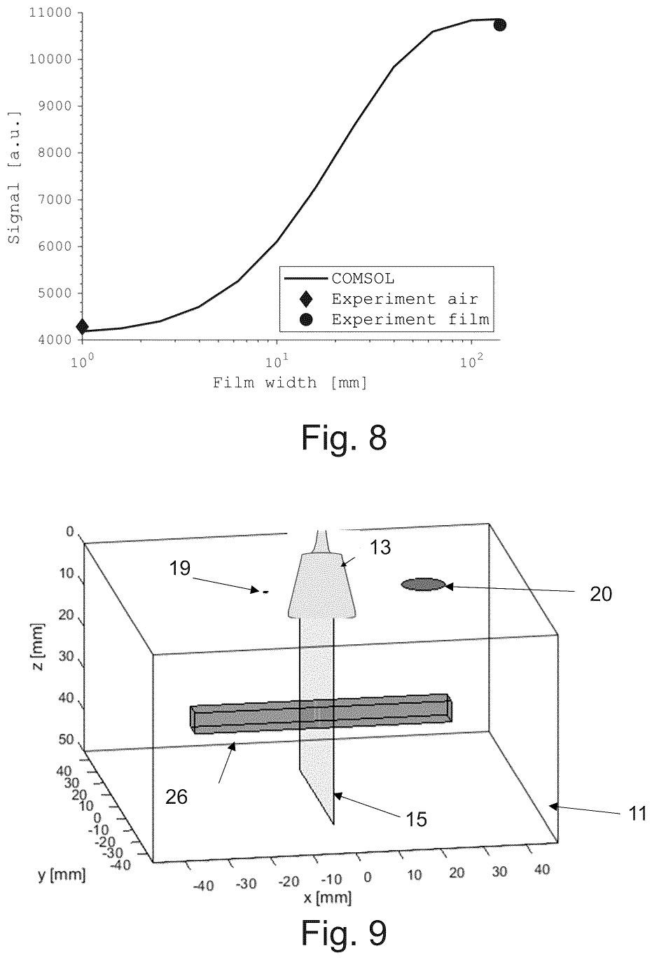

It is of interest to evaluate effect of the size of the area that the light-reflecting film covers around the light injection point. For practical purposes, its desirable to have the light-reflecting boundary cover as little area as possible. FIG. 8 shows the effect on the carrier light signal of variation in size of the reflecting film. This result shows that the signal strength increases with increasing size of the film. However, the returns are diminishing when the film size increases above 2-3 cm. This means that there is no need to have a very large area covered by the reflecting film.

It is also of interest to evaluate the effect of the size of the hole in the reflecting film that is needed to inject the carrier light. Similarly to the previous evaluation, the signal strength increases when the size of the hole decreases. However, the returns are diminishing when the size of the hole decreases below 3-4 mm. Therefore, it is not necessary to have a very small hole.

In the example described in FIG. 5, the light source and detector are placed in transmission mode, i.e., on opposite sides of the light scattering medium. However, the disclosure is not limited to transmission. The relative positions of the light source, light detector, and ultrasound transducer, may be arbitrary. In some cases, it may be practical to place all components on the same side, and perform measurements in reflectance mode. This has been done in a second line of experiments, which setup is depicted in FIG. 9. In this experiment, an optically absorbing inclusion 26 was embedded inside phantom tissue 11. The position of the ultrasound pulse 15 was laterally and longitudinally scanned in a plane perpendicular to the phantom surface with a resolution of 0.75 mm. At each such ultrasound pulse position, a light pulse illuminated the phantom at the input aperture 19 to generate a frequency shifted signal pulse which was resolved using a slow light filter applied after the output aperture 20.

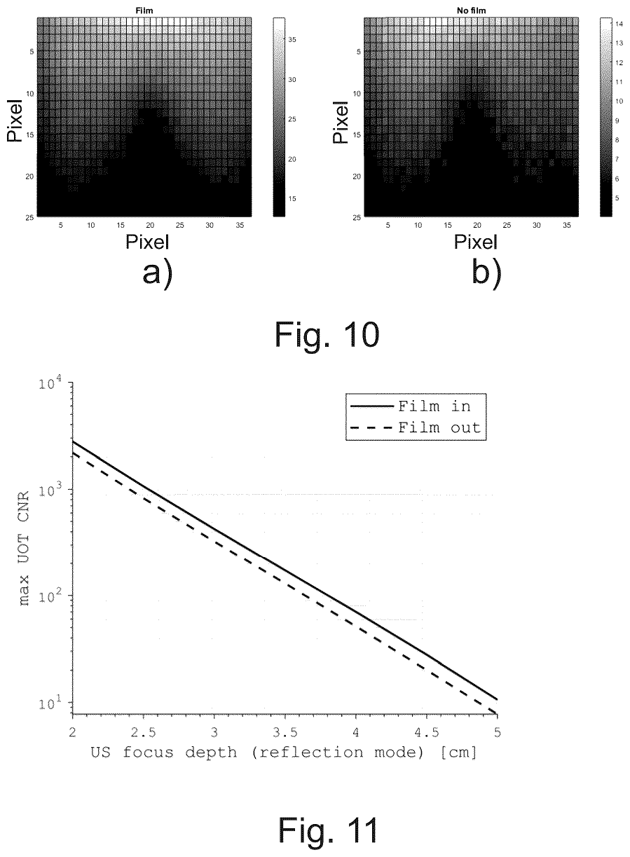

Images were taken of an inclusion with and without a reflecting film as acting as both reflecting member 16 and 17. These images can be seen in FIGS. 10a) and 10b) respectively. The film covered the part of the input surface not occupied by the light input, the ultrasound source and a 1 cm in diameter light guide.

In an additional simulation, the simulation was performed in reflectance mode. To simulate the signal light, a two-step calculation was performed, where first the carrier light was simulated similarly to the simulation presented in FIG. 8. The tagged light was then simulated as a point source at the center of the ultrasound pulse emanating the power F_{US}*K where F_{US} is the carrier fluence at the ultrasound pulse and K is an empirical tagging factor determined experimentally. The scattering and absorption properties of the medium were then chosen to 85% oxygenated muscle tissue for the wavelength 690 nm. The ultrasound pulse was modelled as a sphere with 2 mm radius. The CNR was then calculated with ST1 being the tagged light signal when the local oxygenation around ultrasound pulse is set to 40% and ST2 is the tagged light signal when the local oxygenation is 85%.

In FIG. 11 the CNR for N=200 is shown as function of the depth of the ultrasound pulse, for an oxygenation of 40% in the ultrasound pulse compared to the 85% baseline. It is seen that the CNR clearly increases when the reflecting member is applied, compared to the case with no reflecting member. The CNR increase in the simulated results corresponds well with the CNR increase seen in the experimental results, where the CNR in FIG. 10 is 1.54 for the image taken with reflecting member and 1.02 in the image taken without reflecting member.

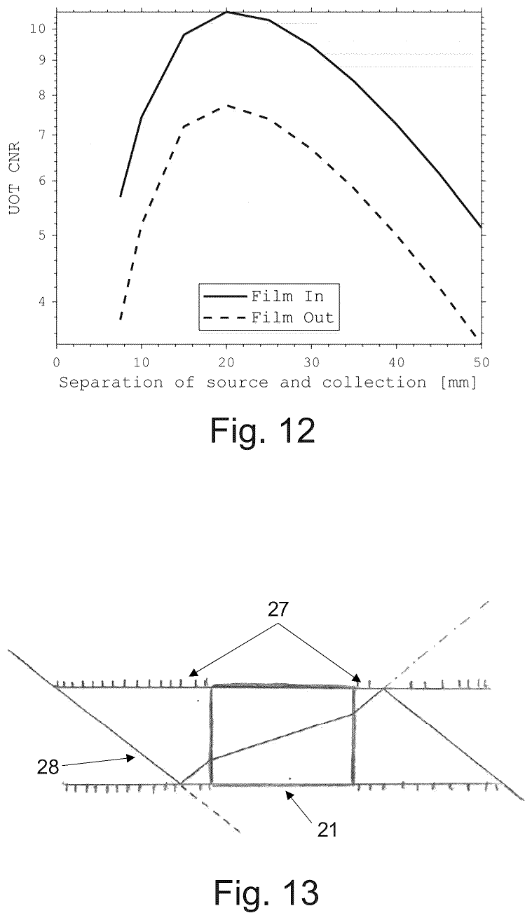

It is of interest to evaluate the distance between the laser injection site and the light detection site, and whether there is an optimal distance in terms of CNR. Since the optical filter suppression of the carrier light is not perfect, it may be optimal to have a certain distance between the source and the detector. FIG. 12 shows the CNR as function of the separation distance of the light source and the detector, for an ultrasound pulse at 5 cm depth, and 80 dB filter suppression. The optimal separation distance in this case is 20 mm. The optimal separation depends generally on the scattering and absorption of the light-scattering medium, the depth of the ultrasound pulse, the tagging factor K, and the filter suppression. Thus, in reflection mode, the optimal measurement geometry can be customised according to these factors.

The advantage of using a reflective member close to or near the light source injection site is not limited to the case of acoustic photon tagging, but it is also advantageous when performing photoacoustic imaging or tomography. In this case, the photoacoustic signal is increased because of the increased light fluence inside the light-scattering medium.

To ensure sufficient signal level in an apparatus as described in the previous, it is important to minimise the light losses between the light collection site and the light detector. The light may be guided to the slow-light filter using a light guide. However, the slow-light filter has to be enclosed inside a cryostatic device and kept at low temperature to function as intended.

The present invention is advantageous to use when there is a medical interest to measure tissue oxygenation deep into the body. For example, the invention may be used in conjunction with ischemic stroke management. In the emergency care setting, it is of importance to rapidly diagnose the presence of an ischemic region in the brain of the patient. A significant portion of ischemic stroke patients undergo thrombectomy to remove the thrombus. In severe cases, the patient cannot be awake during the thrombectomy procedure, which poses the problem that it is not immediately obvious to the surgeon whether the intervention has been successful. In these cases, monitoring of the oxygenation status of the brain would be advantageous as a means of intervention feedback.

Another medical indication where the present invention would be advantageous is for emergency procedures in suspected myocardial infarction, to monitor the oxygenation status of the heart muscle.

Another medical indication where the present invention would be advantageous is in sports medicine, to evaluate oxygen uptake and metabolism in muscles.

Another medical indication where the present invention would be advantageous is in the field of oncology, where the oxygen saturation status may be altered in tumours compared to healthy tissue. For example, there may be necrotic regions in tumours that express low oxygen saturation.

The slow-light filter depends on the use of an optical transition of a dopant ion inside a crystal material. In order to create sufficiently narrow-frequency filters to separate the frequency shifted light from the original frequency light, the linewidth of this transition must be so narrow that the lorentzian tail of the absorption does not overlap between the original laser frequency and the acoustically shifted frequency. The width of these transition lines are limited by the inverse of the coherence time of the transition. If the coherence time is lifetime limited, shorter upper state lifetimes give broader lines. For ions in a crystal lattice, a cause for reduced lifetimes is ion-phonon interaction where phonons are vibrational quanta propagating inside the crystal structure. If the upper state of the ion in the optical transition is close to another state, the ion can transition to this state by the absorption or emission of one or multiple phonons, where events containing more phonons being increasingly less probable. As the maximum energy of phonons is a property of the host crystal, the upper state lifetime may be increased using “soft” crystals where the maximum phonon energy is lower. Phonon transitions in these crystals then require interaction with a higher number of phonons, leading to a reduced transition rate and in turn increased upper state lifetime. The use of soft crystals makes more optical transitions of a given ion viable to use for filtration. This allows more wavelengths to be used to evaluate the oxygenation in tissue using the same crystal.

A given dopant ion can only be used to create filters at wavelengths specific to this ion, e.g. lanthanum. Therefore, using multiple different dopant ions, e.g. lanthanum and europium, enables tailoring of where filters can be created. Multiple ions may be made accessible to the same signal light by doping multiple ions into the same host crystal, i.e. “co-doping”, or by combining different crystals. This combination may be done by either stacking or layering crystals, i.e. “sandwiching”, or melting different crystals to each other, i.e. “splicing”.

The structure created in the absorption profile may be either one or several notch filters, selecting each individual frequency shifted sideband of the original light. It may also be a band stop filter where the only absorption not removed is that at the original frequency. This is of interest since the frequency shifted light in the first sidebands (plus and minus one ultrasound frequency) only accounts for half of the light removed from the original frequency for some ultrasound pulses. This band stop filter does however not have as high slow light effect of the filtered light as a notch filter.

Depending on application and ultrasound pulse, both filters are of interest.

The maximum width of any spectral structure requires that the other ground state/states which the ions are optically pumped to is/are sufficiently far away in frequency. This may be an inherent property of the ion and crystal itself, where the ground state is naturally split into several hyperfine states. In cases this split may be achieved by the application of an electric and/or magnetic field to split a normally degenerate state. The application of an electric and/or magnetic field may also increase the lifetime of the ground states leading to increased filter lifetime. The increased filter lifetime decreases the amount of time during the imaging sequence that must be allotted to filter preparation. A longer filter lifetime thus has the direct consequence of a faster imaging speed. An additional effect when a slow-light filter is created is the simultaneous creation of one or several so called “anti-holes”. Such anti-holes are locations on the crystal absorption profile where the absorption instead is increased. At least one anti-hole is located a frequency distance equal to the ground state split away from the slow-light filter's center frequency. This is caused by the optical pumping as more ions now occupy this state. Applying the correct electric and/or magnetic field may thus, in addition to increasing the hole lifetime, increase the efficiency of a slow-light filter by the placement of the anti-hole at the original laser frequency.

Inside a cryostatic device, a magnetic field may be generated using permanent magnets. This has the advantage of not requiring power supplies or cooling, such as the case for external electromagnets, nor do they introduce any addition thermal load via electric leads, which is the case when using super-conducting magnet coils or a capacitor. Placing the magnets inside the cryostatic device also allows for strong magnetic fields to be generated with smaller magnets compared to them being placed outside. The use of permanent magnets thus allows for a reduction in both size and eases the cooling power of a used cryostatic device.

The shape of the ultrasound pulse also highly effects the spatial resolution of the image and strength of the frequency shifted light. The ultrasound pulse can be chirped, which together with the slow-light filters allows for multiple spatial points to be probed with the same ultrasound pulse. This will effectively decrease the acquisition time and allow for faster image construction.

By chirped ultrasound pulse it is meant a pulse which has a gradient of center frequency along its temporal and spatial profile. Using a short optical pulse, the frequency shifted light will thus also be chirped where each frequency content will couple to different spatial regions. This frequency content can be temporally separated using a slow-light filter as different frequencies of the filtered light propagate at different speeds. Different times in the readout then corresponds to different probe positions in the tissue.

Multiple simultaneous ultrasound pulses at different frequencies where each pulse frequency may be used to the same effect. If each pulse frequency is matched to a slow light filter of different time delay, multiple points may be imaged with the same optical pulse.

To optimize the detected signal, it is important to collect the signal light from the light-scattering medium 11 and guide it as efficiently as possible to the filter. To achieve this, it is preferable to use a light-guide arrangement, such as a single light guide or a bundle of optical fibres. Where the light-guide arrangement is coupled into the cryostatic device that holds the filter, it is important that the light losses are minimized and the thermal insulation of the cryostatic chamber is maintained. A preferable embodiment that achieves this is depicted in FIG. 13. A hollow light-guide 27 with reflecting walls guides the light from the external light-guide arrangement in and out of the crystal 21 located inside a cryostatic device. This way, a ray 28 that both would not hit the filter and subsequently diverge too much to be imaged on a detector is instead contained by the hollow light-guide 27. With this arrangement, a vacuum can be maintained inside the hollow light guide to maintain thermal integrity. At the same time, the hollow light-guide 27 moves the acting light accepting surface of the filter closer to outside the cryostatic device. As no image needs to be preserved in the optical system, the hollow light-guide 27 allows for minimal light losses to and from the filter.

To present the oxygen saturation data to the user, it is preferable to simultaneously display a conventional ultrasound scan (e.g., a B-mode scan) and an image that represents the oxygen saturation, either on an absolute scale or on a relative scale. The representation of the oxygen saturation may be done as a 2D brightness monochrome image, or, preferably, as a 2D false-colour image. A preferable representation is to overlay the false colour oxygen saturation image on the conventional ultrasound image. In a preferable approach, the oxygen saturation image may be toggled on and off using a software function, or gradually blended onto the ultrasound image.

The present invention has been described above with reference to specific examples. However, other examples than the above described are equally possible within the scope of the disclosure. Different method steps than those described above may be provided within the scope of the invention. The different features and steps of the invention may be combined in other combinations than those described. The scope of the disclosure is only limited by the appended patent claims.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases.

1. A system for light measurements in a subject, said system comprising:
at least one ultrasound transducer;
at least one laser configured for emitting light within a wavelength-range of infrared (IR), visible or ultraviolet light;
at least one optical filter;
at least one light detector; and
at least one light-reflecting member;
wherein said ultrasound transducer is configured for directing at least one ultrasound field with at least one ultrasound frequency to at least one location inside a tissue site of said subject; said laser is configured for directing light with at least one light frequency into said tissue site of said subject; said at least one optical filter is arranged before said at least one light detector for suppressing said light frequency but transmitting light with at least one light frequency that is frequency-shifted by said ultrasound frequency; said at least one light detector detecting said light that is frequency-shifted by said ultrasound frequency; said at least one light-reflecting member is configured to be arranged on a surface of said tissue site of said subject.

2. The system of claim 1, wherein said at least one light reflecting member is configured to be arranged on said surface of said tissue site, thereby increase the amount of light being detected by said detector.

3. The system of claim 1, wherein said at least one light-reflecting member is configured to be arranged around an injection point of said at least one laser.

4. The system of claim 1, wherein said at least one light reflecting member is configured to be arranged around a point where said frequency-shifted light exits said tissue site to said at least one optical filter.

5. The system of claim 1, wherein said at least one light-reflecting member has a hole for said light to entering said tissue site and/or for said frequency shifted light to exit, such as said hole has a diameter of between 3 to 4 mm

6. The system of claim 1, wherein said at least one location inside said tissue site is occupied by at least one ultrasound field, and said laser is configured to direct light into said tissue site so that at least a portion of the emitted light passes through said location.

7. The system of claim 1, wherein said frequency-shifted light carries information only from said at least one location inside said tissue site.

8. The system of claim 1, wherein at least one laser and one ultrasound source are configured for scanning at least one location to attain a map or image of an optical contrast inside said tissue site, such as information about the oxygen saturation in said tissue site.

9. The system of claim 1, wherein said at least one light-reflecting member consist of at least one of a glass or metal mirrors, metal surfaces, light reflective plastics, or surfaces covered with reflective paint.

10. The system of claim 1, wherein said at least one optical filter is a slow-light filter structure.

11. The system of claim 10, wherein said at least one slow-light filter structure comprises two different absorption lines to construct filters at two wavelengths.

12. The system of claim 10, wherein said at least one slow-light filter structure comprising a host crystal.

13. The system of claim 12, wherein said host crystal either uses a single ion, be co-doped with multiple ions or be comprised of multiple different crystals spliced or sandwiched together.

14. The system of claim 10, wherein said at least one of slow-light filter structure comprises a notch filter configuration, blocking all light except the frequency shifted light.

15. The system of claim 10, wherein said at least one slow-light filter structure comprises a band stop filter configuration, blocking only the original frequency light.

16. The system of claim 10, wherein an electric and/or magnetic field creating element configured to affect lifetimes of said at least one slow-light filter structure.

17. The system of claim 16, wherein said electric and/or magnetic field is configured to position an anti-hole of said at least one slow-light filter structure at an original laser frequency of said laser.

18. The system of claim 16, wherein said magnetic field creating element comprises at least one permanent magnet.
